(19) United States
(12) Patent Application Publication
(10) Pub. No.: US 2021/0369134 A1
(43) Pub. Date: Dec. 2, 2021

(54) DEVICE AND METHOD TO MEASURE MERIDIAN IMPEDANCES

(71) Applicant: Wellness Allied Inc, Natick, MA (US)

(72) Inventors: Weihui Li, Natick, MA (US); Tao Zhang, Natick, MA (US)

(21) Appl. No.: 16/888,914

(22) Filed: Jun. 1, 2020

(51) Int. Cl. A61B 5/053; A61B 5/00

(52) U.S. Cl. CPC A61B 5/0532; A61B 5/6807; A61B 5/681; A61B 2560/0475; A61B 5/742; A61B 2562/0215; A61B 5/6806

(57) ABSTRACT

A meridian impedance measurement device and method are provided, including: at least one set of measurement electrodes placed on the wrists or ankles, each set including six measurement electrodes in contact with six corresponding meridians; at least one reference electrode placed on the wrists or ankles, simultaneously being in contact with all the six corresponding meridians; at least one impedance measurement unit; a microprocessor; an impedance display unit; and a storage unit. During measurement, the adjustable voltage output unit delivers voltage to each pair of measurement electrode and reference electrode, the impedance measurement unit collects signals and pass the signals to the microprocessor for calculation. The whole procedure is done automatically and can be built into a wearable device.

### TECHNICAL FIELD

The present invention is related to meridian impedance measurement technique in the medical field.

### BACKGROUND

Up to today, many scientific or clinical workers have conducted a large number of studies to measure skin impedance and proposed some methods and theories to use skin impedance as a way to assess human health conditions.

In 1849, German Dubois-Reymond discovered for the first time that human skin demonstrated electrical activity. In 1878, Hermann and Luchsinger in Switzerland discovered that sweating was an important factor affecting skin impedance (Electrodermal activity or EDA). In 1879, Vigouroux in France discovered that there are correlations between skin impedances and psychological activities. The above studies can be found in Literature 1. U.S. Pat. No. 2,829,638A proposed a method to monitor people's arousal and the emotion change by measuring skin impedance.

In addition to the application of skin impedance in the field of psychology, U.S. Pat. No. 4,895,163A uses a two-electrode measuring system to estimate the body fat percentage. U.S. Pat. No. 7,603,171B diagnoses lung cancer based on skin impedance from selected body areas. Patent application US20150018707A1 makes use of skin impedance to determine body pain severity.

Furthermore, the skin impedance was found to change with the stimulating power frequency. According to Literature 2, the typical skin impedance is 500K Ω/cm². The research in Literature 3 shows that the skin impedance ranges from 10K to 1M Ω/cm² at 1 Hz power. When the frequency is increased to 1 MHz, the skin impedance drops to about 300 Ω/cm².

In 1950s, Japanese Dr. Yoshio Nakatani found that some points on the body have lower impedance than surrounding area when he measured skin impedance on patients. Dr. Yoshio Nakatani called these points as electropermeable points (Literature 4). When connecting these points to lines, the lines surprisingly match the meridians described in Traditional Chinese Medicine books. So, in the following, we will call these lines meridians as shown in FIG. 1.

Based on the above findings, Dr. Yoshio Nakatani and the followers designed meridian impedance measurement devices. The devices generally have two electrodes: reference electrode and measurement electrode. During the measurement, the subject holds the reference electrode (a grip conductor) (Literature 4). The measurement electrode is generally a moistened cotton tip or cotton ball inserted in an ebonite cup with metal conductor inside the cup to close the circuit (FIG. 2). The medical staff holds the measurement electrode and presses on each of the measuring points on the skin. The equivalent circuit of the meridian impedance measurement device is shown in FIG. 3. It measures the impedance between the reference electrode held in the palm and the measurement electrodes. There are three meridians running through the palm: hand Tai Yin lung meridian, hand Shao Yin heart meridian, hand Jue Yin pericardium meridian. The meridians under the measurement electrodes are: hand Tai Yang small intestine meridian, hand Shao Yang triple warmer meridian, hand Yang Ming large intestine meridian, hand Shao Yin heart meridian, hand Jue Yin pericardium meridian, hand Tai Yin lung meridian. Correspondingly, there are 6 meridians to measure on the foot: foot Tai Yin spleen meridian, foot Shao Yin kidney meridian, foot Jue Yin liver meridian, foot Tai Yang bladder meridian, foot Yang Ming stomach meridian, foot Shao Yang gallbladder meridian.

U.S. Pat. No. 3,784,908A proposed a device similar to FIG. 2 except that its measurement electrode is placed on the subject's thumb. The technology proposed by the U.S. Pat. No. 3,971,366A is used to assess the health condition based on the measured impedance value with the rectangular or round reference electrode put on the tips of fingers and toes. Patent TW515276U described an improved meridian impedance measurement device. This design can continuously measure the impedances of multiple meridians and the moistened cotton electrode was replaced with standard electrode pads. But the patent only uses one reference electrode to measure impedances from all the meridians.

Patent CN102805622A uses a five-electrode method. The reference electrode is on a certain body part, such as the forehead or Dazhui acupuncture point on the back. CN102949175A uses a measurement method without a reference electrode. Instead, it uses a non-invasive electromagnetic wave detection device to evaluate the activity of the human meridian. The patent did not describe in detail the specific signal that was collected.

Although the meridian impedance measurement devices mentioned above can qualitatively locate the acupuncture points, but they are not suitable to quantitatively measure the meridian impedance in an efficient way, mainly because: 1) These devices require manually pressing the measurement electrode onto each measurement point, and only one acupuncture point can be measured at a time; 2) Since the human body is in a state of dynamic balance, the data measured a few minutes apart can be very different. With the manual measurement method, the time difference from the first acupuncture point measurement to the last acupuncture point measurement may be apart by minutes. Hence the comparison of the impedances between different acupuncture points are unreliable; 3) During the measurement process, the humidity of the cotton ball continuously decreases, which can cause measurement inconsistency; 4) When the patient holds reference electrode, or the medical staff presses the measurement electrode on measuring points, it is inevitable to have relative movement between the electrodes and the skin. In addition, the force the medical staff pressing the electrode on the skin also varies. They both lead to noises that cannot be ignored; 5) These devices all use only one reference electrode, and sometimes, the reference electrode changes positions during the measurement, for example, the patient switches the reference electrode from left hand to the right hand. They cause inconsistency of measurement; 6) Previous devices cannot be used as wearable devices to measure continuous impedances over many hours or days.

### SUMMARY

In view of the problems of the prior arts, the present invention is proposed.

According to an aspect of the present invention, a meridian impedance measurement device for measuring the impedance on meridians is provided, which is characterized by comprising at least one set of measurement electrodes placed on the wrists or ankles, each set including six measurement electrodes attached to the corresponding meridians; at least one reference electrode paired to each set of measurement electrodes, worn on the wrists or ankles, simultaneously in contact with all the six corresponding meridians; at least one impedance measurement unit for measuring impedance between the measurement electrode and the reference electrode; at least one microprocessor for system control; a meridian impedance display unit for displaying measurement results; and a storage unit for storing measurement results.

Optionally, the reference electrode has a tubular shape or a bracelet shape, suitable to wear on the wrists or ankles, and is in contact with all the meridians to be measured by the corresponding set of measurement electrodes.

Optionally, the meridian impedance measurement device is worn on the wrists when in use, and the six corresponding meridians are hand Tai Yang small intestine meridian, hand Shao Yang triple warmer meridian, hand Yang Ming large intestine meridian, hand Shao Yin heart meridian, hand Jue Yin pericardium meridian, and hand Tai Yin lung meridian.

Optionally, the meridian impedance measurement device is worn on the ankles when in use, and the six corresponding meridians are foot Yang Ming stomach meridian, foot Shao Yang gallbladder meridian, foot Tai Yang bladder meridian, foot Tai Yin spleen meridian, foot Jue Yin liver meridian and foot Shao Yin kidney meridian.

Optionally, the meridian impedance measurement device is a wrist bracelet, a watch, a glove, an ankle bracelet, or a foot sock.

Optionally, the meridian impedance measurement device is a smart watch. The measurement electrodes, the reference electrode, and the wires are embedded in the watch strap or back of the watch case. The impedance measurement unit, the microprocessor, the impedance display unit, and the storage unit are assembled inside the watch case.

Optionally, the reference electrode has a tubular or bracelet shape, and is made of a ductile material.

Optionally, the reference electrode includes a plurality of pieces, and each piece is connected by wires or other conductive materials. When in use, each piece is arranged to be in touch with corresponding meridian.

Optionally, the meridian impedance measurement device includes four sets of measurement electrodes and four reference electrodes, the two sets of measurement electrodes and two paired reference electrodes are worn on two wrists respectively, and the other two sets of measurement electrodes and two paired reference electrodes are worn on the ankles.

Optionally, all or part of the four sets of measurement electrodes and the paired reference electrodes share one impedance measurement unit.

Optionally, the four sets of measurement electrodes and the four paired reference electrodes have their independent impedance measurement units.

Optionally, each set of measurement electrodes and the paired reference electrode are built on a tubular carrier, and the tubular carrier is worn on the wrists or ankles when in use.

Optionally, the measurement electrodes and the reference electrode are silver/silver chloride electrodes.

Optionally, the measurement electrodes and the reference electrodes are semi-dry polymer electrodes.

Optionally, the measurement electrodes and the reference electrodes are polarizable electrodes.

Optionally, the measurement electrodes and the reference electrodes are non-polarizable electrodes.

Optionally, the meridian impedance measurement device, if measuring only one channel of impedance, includes an amplification filter circuit and an A/D converter; if measuring multiple channels of impedances, further includes a multiplexer.

Optionally, the impedance measurement unit applies an AC power source to measure the AC characteristics of the meridian, or a DC power source to measure the DC characteristics of the meridian.

Optionally, the impedance measurement unit uses a dedicated impedance measurement chip.

Optionally, the display unit is local or remote.

Optionally, the storage unit is local or remote.

According to another aspect of the present invention, a meridian impedance measurement device to evaluate the meridian's characteristics is provided, comprising: at least one set of N measurement electrodes worn on the wrists and/or ankles, and each in contact with N corresponding meridians; at least one reference electrode worn on the wrists and/or ankles, and in contact with the corresponding N meridians simultaneously; at least one impedance measurement unit to collect and measure electrical signals for the calculation of meridian impedance between the measurement electrodes and the reference electrode; a microprocessor to control the applying of voltage/current to different meridians between the measurement electrodes and reference electrode, to control the measurement of meridian impedance by the impedance measurement unit, and to control the storing of the meridian impedance to the storage unit; a display unit to display the measurement results; and a storage unit for storing the measurement results; N equals or is greater than 2.

According to another aspect of the present invention, a measurement method for measuring the meridian impedance of a human body on the wrists and/or the ankles is provided, comprising: voltage/current applying step, that is to apply voltage/current between the measurement electrodes and the paired reference electrode worn on the wrists and/or ankles, wherein there are at least one set of N measurement electrodes in contact with N corresponding meridians and one paired reference electrode for each set of measurement electrodes, simultaneously in contact with corresponding N meridians; impedance measurement step, that is to measure the meridian impedance between a measurement electrode and a reference electrode through one impedance measurement unit; multiple channels data acquisition step, that is to control the measurement processes by a microprocessor to repeat the above voltage/current applying step and impedance measurement step on other channels with a multiplexer; data displaying and storing step, that is to display and store impedance values from above measurements; and N equals or is greater than 2.

The above-mentioned embodiments of the present invention improve many aspects of problems with previous meridian impedance measurement devices, and have at least the following advantages: 1) adopt innovated reference electrode, being in contact with multiple (for example, six) meridians, making the measurement results better reflecting meridian impedances and allowing the comparison of impedances between different meridians; 2) use multiple reference electrodes, paired to each set of measurement electrodes worn on wrists or ankles, to reduce the measurement error caused by different pathway the stimulating current has to pass when only one reference electrode is used for all the measurement electrodes on different part of the body; 3) In one embodiment, all four sets of measurement electrodes with four paired reference electrodes have independent impedance measurement unit and microprocessor. Such embodiment can easily design a wearable product to continuously measure impedances on meridians over long period of time; 4) The meridian impedance measurement device of one embodiment of the present invention uses standard electrodes that can be fixed on skin, instead of the electrodes that require manual holding for attachment. This allows the microprocessor to automatically control the measurement of the impedances. It minimizes the related noises caused by motion and inconsistent contact with manual operation. Furthermore, the measurement speed is faster; 5) the impedance measurement device, according to one embodiment of the present invention, adopts standard electrodes, which has the same shape, size, dryness/humidity, and presents relatively consistent contact pressure during measurement. It minimizes the measurement error caused by electrodes.

### BRIEF DESCRIPTION OF THE DRAWINGS

FIG. 1 shows the different meridians seen from the front of the body.

FIG. 2 shows a structural diagram of an exemplary meridian impedance measurement device in the prior art (Literature 4). It includes a cup 210 to insert cotton ball, search conductor (cathode) 220, current meter 230, variable resistor 240, changeover switch 250, battery 260 and grip conductor (anode) 270.

FIG. 3 shows an equivalent circuit in measuring meridian impedance when a grip conductor as the reference electrode is held in the hand or attached to the palm of a hand as in the prior art.

FIG. 4 shows an exemplary application according to an embodiment of the present invention.

FIG. 5 shows a schematic block diagram of an exemplary structure of a meridian impedance measurement device according to an embodiment of the present invention.

FIG. 6 shows a schematic diagram of a meridian impedance measurement device on a wrist according to an embodiment of the present invention.

FIG. 7 shows an equivalent circuit in measuring meridian impedance on a wrist according to an embodiment of the present invention.

FIG. 8 shows a schematic diagram of a meridian impedance measurement device with reference electrode having a plurality of pieces, and each piece being connected by wires or other conductive materials, according to an embodiment of the present invention.

FIG. 9 shows a schematic diagram of a meridian impedance measurement device in the form of a wristwatch according to an embodiment of the present invention.

FIG. 10 shows a schematic diagram of a meridian impedance measurement device in the form of a glove according to an embodiment of the present invention.

FIG. 11 shows a schematic diagram of a meridian impedance measurement device in the form of a sock according to an embodiment of the present invention.

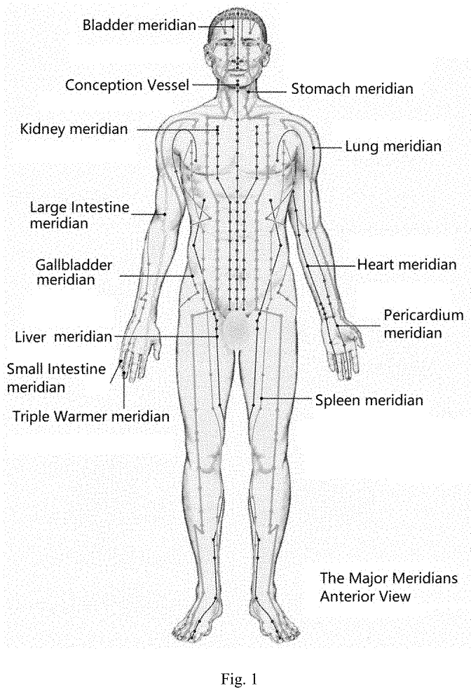

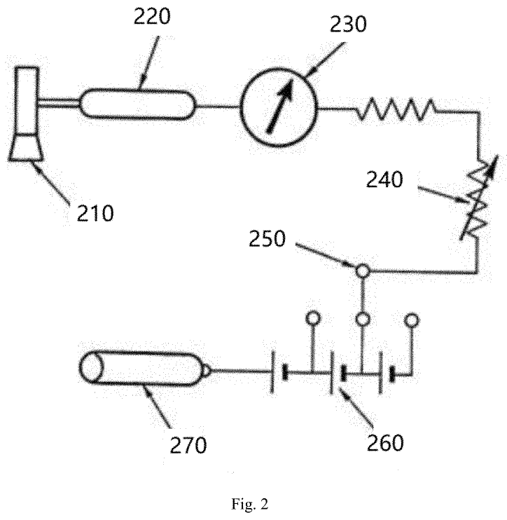

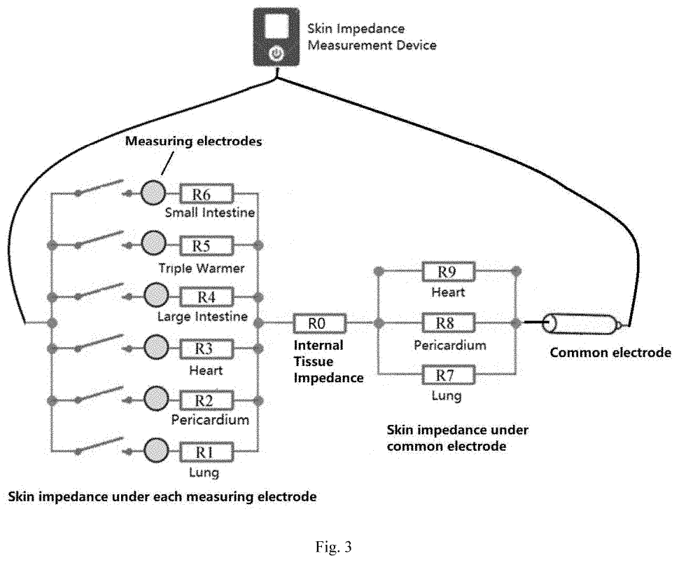

### DETAIL DESCRIPTION

The technical solutions of the present application will be further described below with reference to the accompanying drawings and specific embodiments. It can be understood that the specific embodiments described herein are only used to explain the present application, rather than limiting the present application.

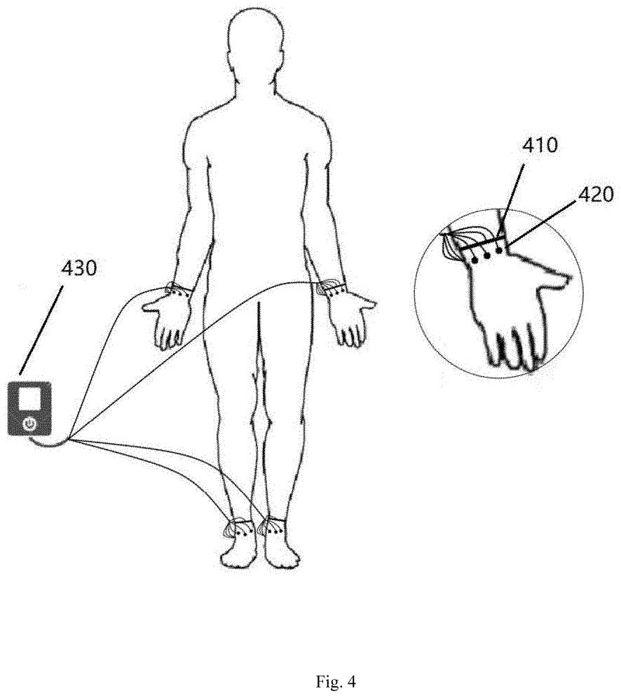

FIG. 4 shows an overall schematic diagram of an application scenario of a meridian impedance measurement device according to an embodiment of the present invention.

In the example shown in FIG. 4, four sets of electrodes, each having six measurement electrodes 420 being in contact with six corresponding meridians and a bracelet shape reference electrode 410 being in contact with the six corresponding meridians simultaneously, are worn on each wrist and ankle. During the measurement, the power supplies voltage/current to the measurement electrodes and the reference electrode, and the meridian impedance measurement device 430 can measure and display the impedances between the measurement electrodes and the reference electrode. Since each measurement result is relative to the pairing reference electrode, the measurement results are standardized and meaningful for data analysis.

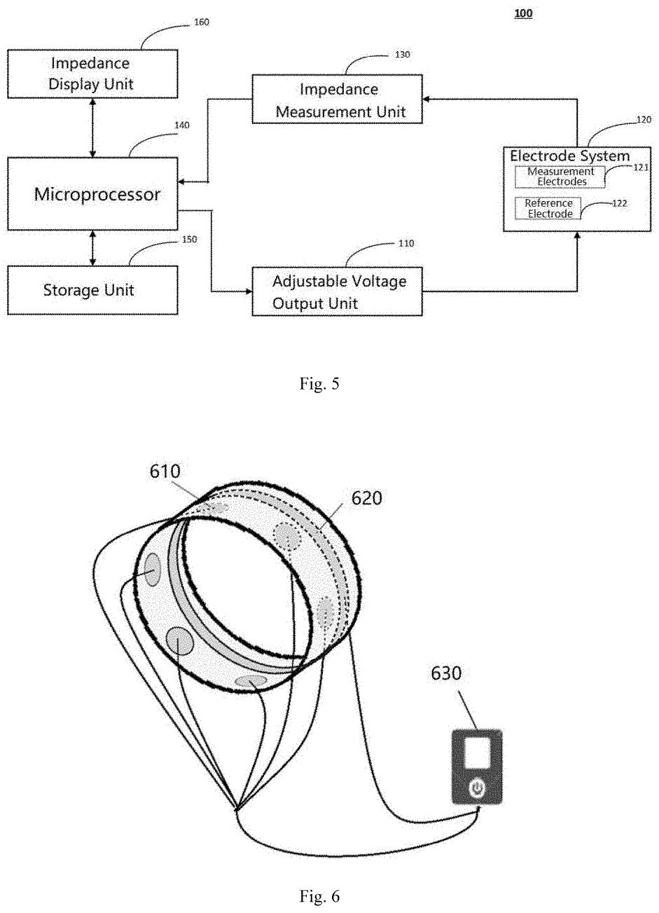

FIG. 5 shows a schematic block diagram of an exemplary structure of a meridian impedance measurement device according to an embodiment of the present invention.

As shown in FIG. 5, the meridian impedance measurement device 100 includes: a adjustable voltage output unit 110, an electrode system 120, an impedance measurement unit 130, a microprocessor 140, a storage unit 150, and a display unit 160. The electrode system 120 includes a measurement electrode 121 and reference electrode 122.

In the case where only one channel of impedance is measured, the meridian impedance measurement device may include an amplification filter circuit and an A/D converter; if multiple channels of impedances are measured, a multiplexer is further included.

Under the microprocessor control, a voltage or current is applied between the reference electrode and the measurement electrode. The impedance measurement unit then acquires the signals, amplifies and filter the signal and passes the signal to the microprocessor to calculate the meridian impedances. For multi-channel impedance measurement, the microprocessor controls the multiplexer circuit to delivery current to different meridian and measure values from them sequentially. The microprocessor then stores and displays the measurement results.

In terms of implementation, the device does not necessarily connect the measurement electrodes and reference electrodes of different limbs to one microprocessor. Independent microprocessor can be used for each set of the electrodes on the limbs. The results can be transmit to a central processor through wireless communication for data processing.

It should be noted that these components can be integrated or distributed. For example, the storage unit can be cloud storage, which can store huge data. With artificial intelligence technology and big data processing, clinical patterns can be discovered.

The power source applying to the electrodes may be an AC current, suitable to examine the AC characteristics of meridian impedance, or a DC current, suitable to examine the DC characteristics of meridian impedance.

The electrode system 120 has measurement electrodes 121 being in contact with each corresponding meridian and a reference electrode 122 being in contact with all the meridians during measurement. The electrode system conducts the voltage/current from the adjustable voltage output unit to the measurement points.

The measurement electrodes 121 may be divided into groups. Each group is placed on the wrists or ankles, and each group includes six measurement electrodes that are in contact with six corresponding meridians.

The reference electrodes 122 pair with the measurement electrodes 121 and are worn on the wrists or ankles. Each reference electrode is in contact with the six corresponding meridians simultaneously.

In one embodiment, the measurement electrodes and the reference electrode are silver/silver chloride electrodes as a representative of non-polarizable electrodes.

In one embodiment, the measurement electrodes and the reference electrodes are metal electrodes as a representative of polarizable electrodes.

These electrodes can be stainless steel electrodes, platinum electrodes, other metal dry electrodes or semi-dry polymer electrodes, which are fixed to the body parts such as wrists and ankles by means of wristbands or ankle bracelet. They can be wet electrodes like Ag/AgCl electrodes, and are fixed on the wrists, ankles or other parts of the body with self-adhesive surface on the electrodes.

A more detailed description will be given later for the arrangement of various measurement electrodes, reference electrodes, and application scenarios of meridian impedance measurement devices.

The impedance measurement unit 130 is configured to measure an electrical signal associated with measurement points, thereby obtaining the impedance between a measurement electrode and a reference electrode.

In one example, if only one channel of impedance is measured, the impedance measurement unit 130 may include an amplification filter circuit and an A/D converter; if multiple channels of impedances are measured, in addition to the amplification filter circuit and the A/D converter, the impedance measurement unit 130 further includes a multiplexer.

The microprocessor 140 may be a general-purpose computer processor or an dedicated integrated circuit.

The storage unit 150 stores measurement results and instructions executed by the microprocessor. The storage unit for storing the measurement results is local or remote.

The impedance display unit 160 displays measurement results. The impedance display unit is a local display or a remote display.

The following gives a more detailed description of the arrangement of various measurement electrodes, reference electrodes, and application scenarios of meridian impedance measurement devices.

FIG. 6 shows a schematic diagram of an embodiment of a meridian impedance measurement device on a wrist according to an embodiment of the present invention.

As shown in FIG. 6, the reference electrode is a piece of bracelet shape conductor, integrated to the inner side of a bracelet. The reference electrode is in contact with all the meridians simultaneously.

Six measurement electrodes are also embedded to the inner side of a bracelet. Six measurement electrodes are in contact with each meridian respectively.

As an example of a wrist bracelet, the six corresponding meridians are hand Tai Yang small intestine meridian, hand Shao Yang triple warmer meridian, hand Yang Ming large intestine meridian, hand Shao Yin heart meridian, hand Jue Yin pericardium meridian, and hand Tai Yin lung meridian.

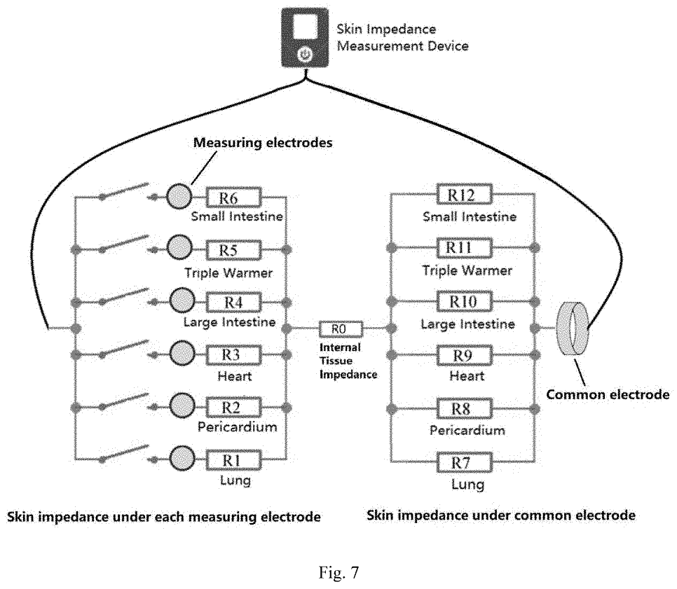

FIG. 7 shows an equivalent circuit of a meridian impedance measurement device according to an embodiment of the present invention.

In the example in FIG. 7, the six measurement electrodes are in touch with corresponding hand Tai Yang small intestine meridian, hand Shao Yang triple warmer meridian, hand Yang Ming large intestine meridian, hand Shao Yin heart meridian, hand Jue Yin pericardium meridian, and hand Tai Yin lung meridian respectively.

The reference electrode is in contact with hand Tai Yang small intestine meridian, hand Shao Yang triple warmer meridian, hand Yang Ming large intestine meridian, hand Shao Yin heart meridian, hand Jue Yin pericardium meridian, and hand Tai Yin lung meridian all together.

In FIG. 7, R1, R2, R3, R4, R5, and R6 represent the impedances under the measurement electrodes on each meridians. R7, R8, R9, R10, R11, and R12 represent the impedance under the reference electrode when it is in contact with all the meridians. R0 is the internal biological tissue impedance.

As shown in FIG. 7, the impedance under the reference electrode is 1/(1/R7+1/R8+1/R9+1/R10+1/R11+1/R12).

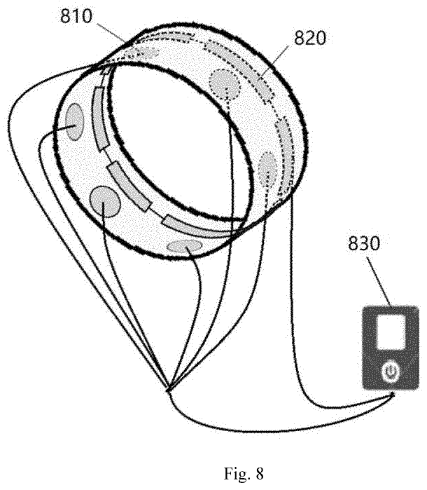

In a specific embodiment, if the bracelet shape reference electrode cannot be made flexible and thus not easy to wear on wrists or ankles, the bracelet shape reference electrode may be split into multiple pieces and connected to each other with wires or other conductive materials, as shown in FIG. 8.

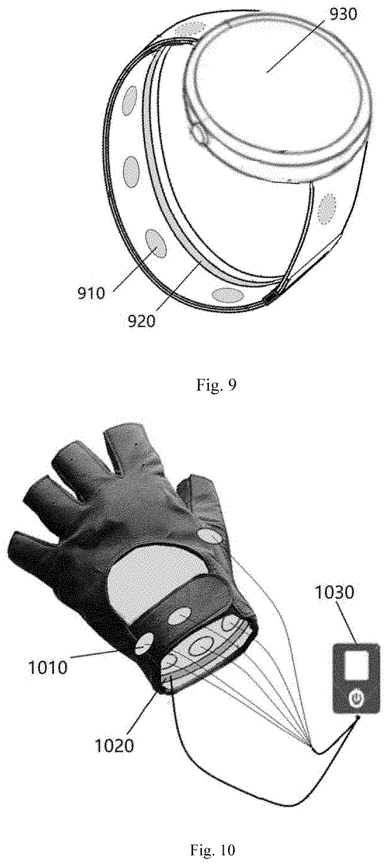

In a specific embodiment, the meridian impedance measurement device can be in the form of a smart watch, with electrodes and wires embedded in the strap, as shown in FIG. 9.

In a specific embodiment, the meridian impedance measurement device can be in the form of an ankle bracelet, with electrodes and wires embedded in the strap and wore on the ankles.

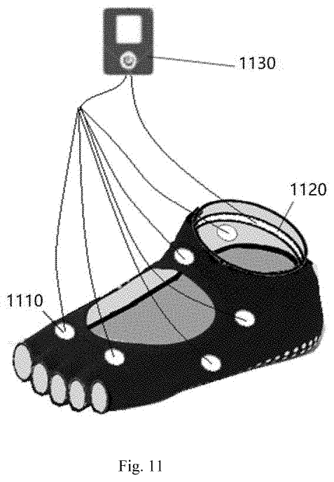

In a specific embodiment, the electrodes can also be embedded in gloves, as shown in FIG. 10.

In a specific embodiment, the electrodes can be embedded in socks, as shown in FIG. 11.

In the example shown in FIG. 4 above, the meridian impedance measurement device includes four sets of measurement electrodes and four reference electrodes. Two sets of the measurement electrodes and the two pairing reference electrodes are worn on two wrists respectively, and the other two sets of measurement electrodes and the two paired reference electrodes are worn on two ankles respectively.

The four sets of measurement electrodes and the four reference electrodes may share one impedance measurement unit, or each may have an independent impedance measurement unit, or two or three of them may share one impedance measurement unit.

According to another embodiment of the present invention, a method to measure meridian impedances is provided, comprising: 1) The microprocessor controls the adjustable voltage output unit to apply voltage/current to a pair of measurement electrode and reference electrode. 2) The microprocessor controls the impedance measurement unit to measure the meridian impedance between the pair of measurement electrode and reference electrode. 3) The microprocessor repeats the steps of applying the voltage/current and measuring the meridian impedances for other pairs of measurement electrodes and reference electrodes by using a multiplexer. 4) Displays and stores impedance values from different meridians.

In the previous embodiment, with the six meridians on each limb, the number of measurement electrodes in each set is set to six, and each reference electrode is set to contact with the six meridians simultaneously. However, according to actual needs, the number of meridians to be measured can be different, and can be more or less than 6, that is, the number of measurement electrodes contained in each set of measurement electrodes can be more or less than 6. The number of meridians being simultaneously contacted with the reference electrodes can be more or less than 6, but is more than or equal to 2.

According to another embodiment of the present invention, a meridian impedance measurement device for measuring meridian impedance is provided, which comprises:

at least one set of measurement electrodes placed on the wrists and/or ankles, each set including N measurement electrodes being in contact with the each of the N meridians;

at least one reference electrode placed on the wrists and/or ankles, paired to each set of measurement electrodes, being in contact with the N meridians simultaneously;

at least one impedance measurement unit for measuring the impedances between the measurement electrodes and the reference electrode;

at least one microprocessor for system controlling;

a display unit for displaying measurement results; and

a storage unit for storing measurement results;

where N is greater than or equal to 2.

The above-mentioned embodiments of the present invention solve the problems that the previous meridian impedance measurement devices have. They have at least the following advantages: 1) The meridian impedance measurement device of the present invention adopted an innovated reference electrode, which is in contact with multiple (for example, six) meridians on the wrists and ankles, thus minimizing the common impedance presented in the measured results and reduced the biased effects when part of the meridians are in contact with the reference electrode; 2) In one embodiment, there is one reference electrode on each of the wrist and ankle. It reduces the measurement error caused by using only one particular acupuncture point or a hand as the reference electrode, which creates different current path for different meridian impedance measurement; 3) In one embodiment, four reference electrodes pairing to four sets of measurement electrodes are used for two wrists and two ankles. They all have their own impedance measurement unit and microprocessor. This type of the embodiment of the present invention is easy to implement as wearable devices to measure continuous values; 4) The measurement electrodes and reference electrodes use standard electrodes that can be fixed on the body instead of electrodes that require manual operation. This allows the measurement to be performed automatically by a microprocessor, which eliminates the need to manually perform the measurements on different parts of the human body. The measurement process is more efficient and also minimizes related errors or noises caused by manual operation; 5) In the meridian impedance measurement device according to the embodiment of the present invention, standard electrodes are used, which have the same shape, size, dryness/humidity, and relatively consistent contact pressure. Therefore, the measurement errors due to electrode variations can be minimized.

The embodiments of the present invention have been described above. The above description is exemplary, not exhaustive, and is not limited to the disclosed embodiments. Many modifications and variations will be apparent to those skilled in the art without departing from the scope and spirit of the embodiments described. Therefore, the protection scope of the present invention shall be subject to the protection scope of the claims.

1. A meridian impedance measurement device for measuring meridian impedances of wrists or ankles, comprising:
at least one set of measurement electrodes placed on the wrists or ankles, and each set includes six measurement electrodes respectively in contact with the six corresponding meridians to be measured;
at least one reference electrode, each reference electrode being paired to a set of measurement electrodes, placed on the wrists or ankles, and simultaneously being in contact with the six corresponding meridians to be measured;
at least one impedance measurement unit for measuring impedances between the measurement electrodes and the reference electrodes;
at least one microprocessor for system control;
an impedance display unit for displaying measurement results; and
a storage unit for storing measurement results.

2. The meridian impedance measurement device according to claim 1, wherein the reference electrode is of a tubular and bracelet shape, suitable to wear on wrists or ankles, with its inside surface in contact with all the six meridians being measured.

3. The meridian impedance measurement device according to claim 1, wherein the device is worn on the wrist during application, and the six corresponding meridians are hand small intestine meridian, hand triple warmer meridian, hand large intestine meridian, hand heart meridian, hand pericardial meridian, and hand lung meridian.

4. The meridian impedance measurement device according to claim 1, wherein the device is worn on the ankles during application, and the six corresponding meridians are foot stomach meridian, foot gallbladder meridian, foot bladder meridian, foot spleen meridian, foot liver meridian, and foot kidney meridian.

5. The meridian impedance measurement device according to claim 1, wherein the device is a wrist bracelet, a watch, a glove, an ankle bracelet, or foot socks.

6. The meridian impedance measurement device according to claim 5, the device is a smart watch, with the measurement electrodes, the reference electrodes and wires being embedded in the watch strap, and the impedance measurement unit, the microprocessor, the impedance display unit, and the storage unit being integrated in the watch case.

7. The meridian impedance measurement device according to claim 1, wherein the reference electrode has tubular and bracelet shape, and is made of a ductile material.

8. The meridian impedance measurement device according to claim 1, wherein the reference electrode comprises a plurality of pieces connected by conductive materials.

9. The meridian impedance measurement device according to claim 1, comprising four sets of measurement electrodes and four reference electrodes: two sets of measurement electrodes and two corresponding paired reference electrodes being put on two wrists, and the other two sets of measurement electrodes and two corresponding paired reference electrodes being put on two ankles respectively.

10. The meridian impedance measurement device according to claim 9, wherein all or part of the four sets of measurement electrodes and four reference electrodes have a common impedance measurement unit.

11. The meridian impedance measurement device according to claim 9, wherein each set of measurement electrodes and corresponding paired reference electrode have independent impedance measurement unit.

12. The meridian impedance measurement device according to claim 1, wherein each set of measurement electrodes and the paired reference electrode are placed on a common tubular carrier, to be worn on the wrists or ankles during application.

13. The meridian impedance measurement device according to claim 1, wherein the measurement electrodes and the reference electrodes are silver/silver chloride (Ag/AgCl) electrodes.

14. The meridian impedance measurement device according to claim 1, wherein the measurement electrodes and the reference electrodes are polarizable electrodes.

15. The meridian impedance measurement device according to claim 1, comprising an amplifying filter circuit and an A/D converter when only one channel of impedance is measured; or further comprising a multiplexer besides an amplifying filter circuit and an A/D converter if multiple channels of impedance are measured.

16. The meridian impedance measurement device according to claim 1, wherein the adjustable voltage output unit delivers an AC source to measure the AC characteristics of the meridian impedance, or a DC source to measure the DC characteristics of meridian impedance.

17. The meridian impedance measurement device according to claim 1, wherein the impedance measurement unit uses a dedicated impedance measurement chip.

18. The meridian impedance measurement device according to claim 1, wherein the display unit and storage unit are local or remote.

19. A device for measuring meridian impedance to evaluate the meridian's characteristics, comprising:
at least one set of measurement electrodes placed on the wrists and/or ankles, and each set including N measurement electrodes in contact with N corresponding meridians respectively;
at least one reference electrode worn on the wrists and/or ankles, and being in contact with all the corresponding N meridians simultaneously;
at least one impedance measurement unit to collect and measure electrical signals for the calculation of meridian impedance between the measurement electrodes and the reference electrodes;
a microprocessor to control applying of voltage/current to different meridians to measure meridian impedance between different measurement electrodes or reference electrodes;
a display unit to display the measurement results; and
a storage unit for storing measurement results,
wherein N equals or is greater than 2.

20. A measurement method for measuring the meridian impedance of a human body on the wrists and/or the ankles, comprising:
applying voltage/current between measurement electrodes and reference electrodes worn on the wrists and/or ankles, wherein there are at least one set of measurement electrodes placed on the wrists and/or ankles, and each set includes N measurement electrodes in contact with N corresponding meridians and there is at least one paired reference electrode, each reference electrode being paired to a set of measurement electrodes, and simultaneously in contact with corresponding N meridians;
measuring the meridian impedance between the measurement electrodes and a reference electrode through at least one impedance measurement unit;
repeating the above voltage/current applying step and impedance measurement step controlled by a microprocessor with a multiplexer, thus obtaining these N meridian impedances between N measurement electrodes or the paired reference electrodes;
displaying and storing impedance values from different meridians; and
N equals or is greater than 2.
